(19) United States
(12) Patent Application Publication
(10) Pub. No.: US 2026/0325012 A1
(43) Pub. Date: Oct. 22, 2026

(54) METHOD FOR ESTIMATING A TRAJECTORY OF A POINT OF INTEREST

(71) Applicant: SYSNAV, VERNON (FR)

(72) Inventors: Alexis TRICOT, PARIS (FR); Dimitri LOZEVE, PARIS (FR); Mélanie ANNOUSSAMY, Longjumeau (FR)

(21) Appl. No.: 19/115,690

(22) PCT Filed: Nov. 20, 2023
(86) PCT No.: PCT/FR2023/051822

§ 371 Date: Mar. 26, 2025

(30) Foreign Application Priority Data
Nov. 18, 2022 (FR) FR2212003

(51) Int. Cl. A61B 5/11; G01P 15/18

(52) U.S. Cl. CPC A61B 5/1122; G01P 15/18

(57) ABSTRACT

This method for estimating a trajectory, over a displacement interval, of a point of interest belonging to a limb of an individual comprises the estimation (1320) of the evolution, during the displacement interval, of a speed of a measurement point attached to the point of interest, the estimation (1310) of the evolution of an orientation of the measurement point during the displacement interval, and the estimation of the trajectory of the point of interest during the displacement interval from the estimated speed and orientation of the measurement point.

### FIELD OF INVENTION

The present invention relates to the field of estimating the evolution of the linear speed of a point from measurements carried out using an inertial device.

The invention also relates to the estimation of the trajectory of a point of interest belonging to a limb of an individual from measurements carried out by means of an inertial device, and the use of such an estimation to evaluate and analyze a force exerted by said limb, in particular when this limb is an upper limb.

### TECHNOLOGICAL BACKGROUND

For an individual, moving their upper limbs depends on the use and coordination of several muscles. Analysis of an individual's movements can therefore be used to characterize their fitness level, and in particular the muscular strength that the latter is capable of developing. This muscular strength is likely to vary depending on many factors such as the individual's fitness level, their age, their physical training, or taking a treatment affecting their muscular efficiency.

The evolution of an individual's movements over time can be representative of the evolution of their physical fitness, particularly in the case of an individual suffering from a neuromuscular or neurodegenerative disease. These diseases, such as Duchenne Muscular Dystrophy (DMD), result in a reduction in the individual's muscular capacities as the disease progresses. This leads to a loss of motor skills in the patients concerned.

WO 2019/243609 discloses a method for analyzing the stride of a walking pedestrian, intended to characterize the evolution of his fitness, from measurements taken using an inertial device attached to the pedestrian's ankle. Such a method, which is minimally invasive, finds a very particular application in the tracking of neuromuscular or neurodegenerative diseases. However, it is only suitable for tracking such diseases as long as the individual retains sufficient motor function in his lower limbs. When the individual is no longer able to walk, typically because the disease is at an advanced stage, an alternative method is necessary to allow the disease to be tracked.

A consensus has thus emerged in the scientific community on the need to be able to precisely quantify the motor function of the upper limbs of individuals suffering from neuromuscular or neurodegenerative diseases. This consensus has led to the emergence of several methods for evaluating this motor function.

The vast majority of these evaluation methods consist of conventional methods based on functional scales evaluated by professional clinicians in controlled environments. The individual is typically invited to a hospital where they are asked to perform certain movements and, based on the success or failure of these movements, the clinician assigns a motor score. These methods include, for example:
the Brooke scale (Brooke, M H et al. “Clinical investigation in Duchenne dystrophy: 2. Determination of the “power” of therapeutic trials based on the natural history.” Muscle & nerve vol. 6, 2 (1983): 91-103. doi: 10.10002/mus.880060204),
the revised upper limb module (better known by the acronym RULM) (Mazzone, Elena S et al. “Revised upper limb module for spinal muscular atrophy: Development of a new module.” Muscle & nerve vol. 55, 6 (2017): 869-874. doi: 10.10002/mus.25430), and
the performance of upper limb module (better known by the acronym PULM) (Mayhew, Anna G et al. “Performance of Upper Limb module for Duchenne muscular dystrophy.” Developmental medicine and child neurology vol. 62, 5 (2020): 633-639. doi: 10.1111/dmcn.14361).

However, these methods have several disadvantages: they are restrictive for the patient, requiring them to travel to a hospital, and their results may vary depending on the patient's state of health on the day of the evaluation and the evaluator.

Recently, other methods have emerged, based on the use of information technologies. Most use the analysis of data recorded in a controlled environment, typically in a hospital, using an optical capture system under the control of a health professional, as described for example in Han, J. J. et al. “Reachable workspace and performance of upper limb (PUL) in duchenne muscular dystrophy.” Muscle & nerve vol. 53 (2016): 545-554. doi: 10.10002/mus.24894. However, these methods suffer from most of the disadvantages already identified for conventional methods, in particular because they are restrictive for the patient and produce results that vary depending on the patient's fitness level on the day of the evaluation.

To solve these problems, solutions have been sought to enable the evaluation of the motor function of the upper limbs over long acquisition times, in the daily life of patients, most often by means of measurements carried out by inertial devices attached to the upper limbs. These inertial devices must meet particularly demanding constraints of weight, size and autonomy to allow their portability, they are composed of MEMS type sensors. However, such sensors have a significant time drift which greatly complicates the tracking of movements using the measurements returned by these sensors.

To circumvent this difficulty, it was proposed in WO 2017/129890 to assess the motor function of the upper limbs only for very specific movements, when the elbow is placed motionless on a surface and the movement of the wrist, to which the inertial device is attached, follows an arc of a circle centered on the elbow. However, this solution is not entirely satisfactory, since many movements of the upper limbs are not taken into account in this evaluation of the motor function.

It has also been proposed in El-Gohary, Mahmoud, and James McNames. “Shoulder and elbow joint angle tracking with inertial sensors.” IEEE transactions on bio-medical engineering vol. 59, 9 (2012): 2635-41. doi: 10.11009/TBME.2012.2208750 to combine a kinematic model of the upper limb with measurements acquired by two inertial devices positioned one on the arm, the other on the forearm, to compensate for sensor drift. This solution, which uses several inertial devices, is however poorly suited to daily use, because it is too invasive.

Finally, it was proposed in Villeneuve, Emma et al. “Reconstruction of Angular Kinematics From Wrist-Worn Inertial Sensor Data for Smart Home Healthcare.” IEEE Access, 5 (2017): 2351-2363. doi: 10.11009/access.2016.2640559 to estimate the kinematics of an individual's wrist from measurements provided by simple accelerometers worn at the wrist, using a particle filter and imposing constraints related to the respect of a biomechanical model of the individual's upper limb. However, if this method allows to estimate the kinematics of the wrist without using a gyrometer, it does not solve the problem of the temporal drift of the sensors.

Other difficulties encountered in evaluating upper limb motor function using inertial devices are that upper limb movements are much more varied and difficult to recognize than lower limb movements and that, unlike the latter, upper limb movements are often related not only to their own muscular activity, but also to muscular activity in other parts of the body, particularly the torso or lower limbs.

### DISCLOSURE OF THE INVENTION

One objective of the invention is to enable the tracking of an individual's physical fitness over time. Another objective is to enable a good estimation, in an uncontrolled environment and over a long period, of the physical forces made by an individual's upper limb for various movements. Other objectives are to minimize the invasive nature of the devices used to perform this estimation, and to improve the quality of the estimations deduced from the measurements of an inertial device.

For this purpose, the invention relates, according to a first aspect, to a method for estimating the evolution of a linear speed of a measurement point during a displacement interval, the method being implemented by a data processing unit and comprising the following steps:
determining an initial speed and a final speed of the measurement point respectively at the start and at the end of the displacement interval,
estimating the evolution of a speed of the measurement point during the displacement interval, said estimation comprising:
   forward integration of displacement parameters of the measurement point acquired by an inertial device during the displacement interval, taking as the initial value of the speed the value of the initial speed,
   the backward integration of the displacement parameters, taking as the initial value of the speed the value of the final speed, and
   the calculation of the estimated speed by merging the backward and forward integrations.

The invention also relates, according to a second aspect, to a method for estimating a trajectory, over a displacement interval, of a point of interest belonging to a limb of an individual, the method being implemented by a data processing unit and comprising the following steps:
estimating the evolution, during the displacement interval, of a speed of a measurement point attached to the point of interest,
estimating the evolution of an orientation of the measurement point during the displacement interval, and
estimating the trajectory of the point of interest during the displacement interval from the estimated speed and orientation of the measurement point.

According to particular embodiments of the invention, the trajectory estimation method also has one or more of the following features, taken in isolation or in any technically possible combination(s):
the individual is a human being;
the limb is an upper limb of the individual;
the point of interest is a point of a forearm of said upper limb;
the limb is a lower limb of the individual;
the point of interest is a point of a leg of said lower limb;
the evolution of the speed of the measurement point during the displacement interval is estimated by means of a method according to the first aspect;
the estimation of the trajectory comprises the following steps:
   calculating multiple candidate trajectories by applying the estimated speed and orientation to different pairs of starting and finishing configurations of a biomechanical model of the limb,
   evaluating the compatibility of each candidate trajectory with the biomechanical model, and
   selecting the candidate trajectory having the best compatibility;
the point of interest is a point of a forearm belonging to an upper limb of the individual, each starting configuration of the biomechanical model of the limb being defined by a pair of starting angles formed by a first starting angle between the forearm and the vertical and a second starting angle between the arm and the vertical and each finishing configuration of the biomechanical model of the limb being defined by a pair of finishing angles formed by a first finishing angle between the forearm and the vertical and a second finishing angle between the arm and the vertical;
for each starting or finishing configuration of the biomechanical model of the limb:
   the first starting angle, respectively finishing angle, is deduced from the orientation of the measurement point at the beginning, respectively at the end, of the displacement interval, and
   the second starting, respectively finishing, angle is less than the first starting, respectively finishing angle;
there is, for each value of second starting angle smaller than the first starting angle and each second finishing angle value within a predetermined interval, a pair of starting and finishing configurations for which a candidate trajectory is calculated;
the predetermined interval is constituted by an interval comprised between 0° and the value of the first finishing angle, or by an angular interval around an angle βₜ verifying the following relation:

$$L ( cos ( {β}_{i} ) - cos ( {β}_{t} ) ) = ( {\int }_{{t}_{i}}^{{t}_{f}} \vec{V ( t )} dt - l ( \vec{{O}_{f}} - \vec{{O}_{l}} ) ) \cdot \vec{Z}$$

in which:
   L is the length of the arm of the upper limb,
   βᵢ is the value of the second starting angle in the starting configuration belonging to the same pair as the finishing configuration,
   tᵢ is the time of the start of the displacement interval,
   t_{f} is the end time of the displacement interval,
   \vec{V(t)} is the speed vector of the measurement point at each time t of the displacement interval,
   l is the distance between the elbow of the upper limb and the measurement point,
   \vec{O_{f}} is a unit vector giving the orientation of a main axis of the forearm at the end of the displacement interval,
   \vec{Oᵢ} is a unit vector giving the orientation of the main axis of the forearm at the beginning of the displacement interval,
   \vec{Z} is a unit vector giving the vertical orientation,
the vectors \vec{V(t)}, \vec{O_{f}}, \vec{Oi} and \vec{Z} being expressed in the same inertial reference frame;
the angular interval extends over less than 20°, preferably over less than 15°, for example over substantially 10°; and
the calculation of each candidate trajectory comprises the following steps:
   forward integration of the estimated speed, taking as initial position the position of the measurement point in the starting configuration,
   backward integration of the estimated speed, taking as initial position the position of the measurement point in the finishing configuration, and
   calculation of the candidate trajectory by merging the backward and forward integrations;

The invention also relates, according to a third aspect, to a method for evaluating a force exerted by a limb of an individual, the method being implemented by a data processing unit and comprising the following steps:
estimating a trajectory of a point of interest belonging to the limb over at least one primary displacement interval by means of a method according to the second aspect,
evaluating, from the estimated trajectory and speed evolution, a primary force exerted by the limb during the or each primary displacement interval.

According to particular embodiments of the invention, the force evaluation method also has one or more of the following features, taken in isolation or in any technically possible combination(s):
the method comprises the following additional steps:
   deducing, from the displacement parameters acquired during the primary displacement interval, a rotational component of a primary kinetic energy acquired by at least a portion of the limb during the primary displacement interval, the primary force being a function of said rotational component of the primary kinetic energy,
   determining a relationship between the primary force and the rotational component of the primary kinetic energy,
   estimating the evolution of an orientation of a measurement point attached to the point of interest during at least one secondary displacement interval,
   deducing, from displacement parameters acquired by the inertial device during a secondary displacement interval, a rotational component of a secondary kinetic energy acquired by said at least one portion of the limb during the secondary displacement interval,
   evaluating, from the determined relationship and the rotational component of the secondary kinetic energy, a secondary force exerted by the limb during the or each secondary displacement interval;
the method comprises, for each of the primary and secondary displacement intervals:
   estimating the evolution, during the primary or secondary displacement interval, of a speed of a measurement point attached to the point of interest,
   calculating several candidate trajectories by applying the estimated speed and orientation to different pairs of starting and finishing configurations of a biomechanical model of the limb,
   evaluating the compatibility of each candidate trajectory with the biomechanical model, and
   verifying, for each primary or secondary displacement interval, the acceptable nature of the compatibility of at least one candidate trajectory,
the trajectory of the point of interest being estimated only when at least one candidate trajectory has acceptable compatibility; and
the evolution of the speed of the measurement point during the primary or secondary displacement interval is estimated by means of a method according to the first aspect.

The invention also relates, according to a fourth aspect, to a method for analyzing a force exerted by a limb of an individual, the method being implemented by a data processing unit and comprising the following steps:
a) estimating, for several intervals of displacement of a point of interest of the limb occurring during a predetermined period, a force exerted by said limb by means of a method according to the third aspect,
b) determining the value of at least one statistical quantity, calculated on the set formed by the estimated forces, representative of a state of fitness of the individual.

According to a particular embodiment of the invention, the force analysis method also has the following feature:
the method comprises repeating steps a) and b) for several predetermined periods and observing the evolution of the or each statistical quantity across the different predetermined periods.

The invention also relates, according to a fifth aspect, to a method for tracking the fitness level of an individual, comprising the analysis of a force exerted by a limb of the individual by means of an analysis method according to the fourth aspect and the deduction of a fitness level of the individual from said analysis.

According to a particular embodiment of the invention, the tracking method also has the following feature:
the method comprises administering a treatment to the individual, and evaluating an effectiveness of the treatment from the deduced fitness status.

Finally an object of the invention is, according to respectively a fifth, a sixth and a seventh aspects of the invention:
equipment comprising an inertial device for acquiring displacement parameters of a measurement point during at least one displacement interval, and a data processing unit configured to estimate, from the displacement parameters acquired by the inertial device, by implementing a method according to any one of the first, second and third aspects, at least one of: the evolution of a speed of the measurement point during the displacement interval, a trajectory, over the displacement interval, of a point of interest attached to the measurement point, and a force exerted by a limb of an individual to which the point of interest belongs;
a computer program product comprising code instructions for implementing a method according to any of the first, second, third, fourth and fifth aspects when said program is executed by a computer; and
a storage medium readable by computer equipment on which is stored a computer program product comprising code instructions for implementing a method according to any one of the first, second and third aspects when said program is executed by a computer.

### BRIEF DESCRIPTION OF THE FIGURES

Other features and advantages of the invention will become apparent upon reading the following description, given only by way of example and with reference to the appended drawings, in which:

FIG. 1 is a diagram of equipment according to an exemplary embodiment of the invention,

FIG. 2 is a diagram of a measuring device of the equipment of FIG. 1,

FIG. 3 is a diagram of a movement sensor of the equipment of FIG. 1,

FIG. 4 is a diagram illustrating a force analysis method implemented by the equipment of FIG. 1,

FIG. 5 is a diagram detailing a step of the method of FIG. 4,

FIG. 6 is a diagram detailing a first sub-step of the step in FIG. 5,

FIG. 7 is a diagram detailing a second sub-step of the step in FIG. 5,

FIG. 8 is a diagram detailing a third sub-step of the step in FIG. 5,

FIG. 9 is a diagram of a biomechanical model of an upper limb used for implementing the method of FIG. 4, and

FIG. 10 is a diagram showing a pair of starting and finishing configurations of the biomechanical model of FIG. 9.

### DETAILED DESCRIPTION OF AN EXEMPLARY EMBODIMENT

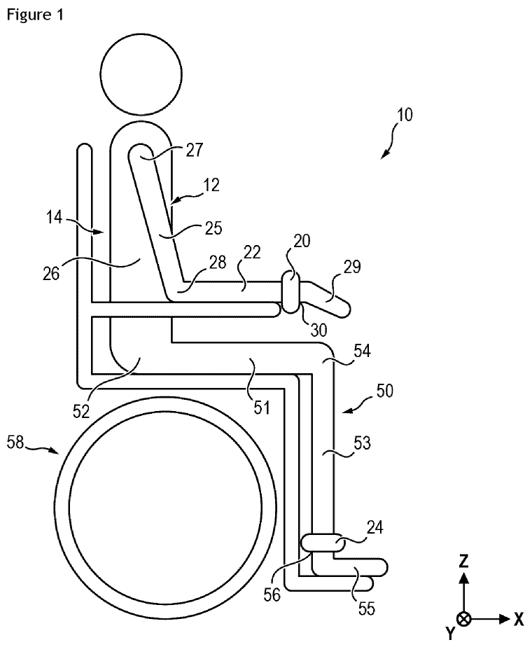

The equipment 10 shown in FIG. 1 is intended for the evaluation and analysis of the forces exerted by the muscles of an upper limb 12 of an individual 14. “Individual”, here and hereinafter, means a human being. For this purpose, the equipment 10 comprises a measuring device 20 attached to a forearm 22 of the upper limb 12. In the example shown, it also comprises a movement sensor 24 intended to capture the displacement of at least one lower limb of the individual 14.

It is recalled here that, anatomically, an upper limb such as the upper limb 12 is made up of three segments: the arm 25, articulated to the torso 26 by the shoulder 27, the forearm 22, articulated to the arm 25 by the elbow 28, and the hand 29, articulated to the forearm 22 by the wrist 30. Here and hereinafter, it is in this sense that these terms are used, the term “arm” being in particular used to designate only the segment of the upper limb between the elbow and the shoulder and not, as in common language, the entire upper limb.

The measuring device 20 is secured to the forearm 22 of the upper limb 12, that is to say that it has, in the terrestrial reference frame, a movement substantially identical to that of the forearm 12. It is in particular placed between the elbow 28 (excluded) and the wrist 30 (included), for example, as shown, substantially on the wrist 30.

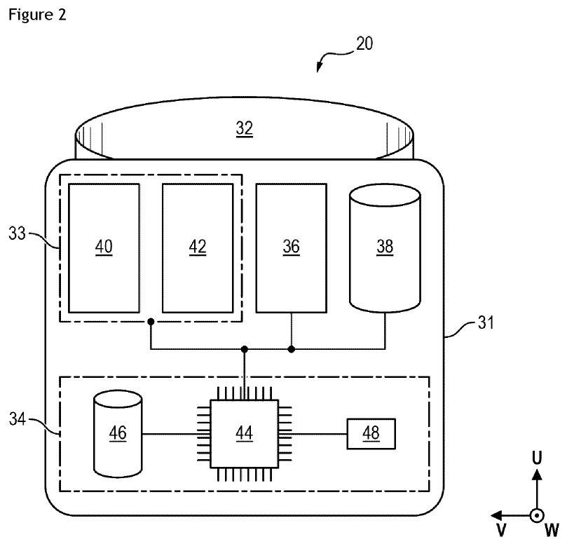

With reference to FIG. 2, the measuring device 20 comprises a support 31 and an attachment element 32 for attaching the support 31 to the forearm 22. It also comprises an inertial device 33 and a data processing unit 34 mounted on the support 31. In the example shown, it further comprises a communication system 36, typically a wireless communication system, mounted on the support 31 for communication of the measuring device 20 with the second measuring device 24 and/or an external device such as a mobile terminal (not shown), for example a multifunction mobile, or a remote server (not shown). Optionally, it further comprises a storage module 38 mounted on the support 31.

The support 31 is typically constituted by a casing.

The attachment element 32 is here constituted by a bracelet, for example with hook-and-loop strip, adapted to encircle the forearm 22 and allow the integral connection. Alternatively (not shown), the attachment element 32 is constituted by any element allowing a connection integral with the forearm 22.

The inertial device 33 comprises a gyrometer 40 for measuring an angular speed of the measuring device 20 according to a system of three orthogonal axes defining a moving reference frame (Rₘ) integral with the measuring device 20, that is to say measuring the three components of an angular speed vector in said moving reference frame (Rₘ). It is thus understood that the gyrometer 40 is typically made up of a set of three gyrometers each associated with one of the three axes, in particular in tri-axis (that is to say each capable of measuring one of the three components of the angular speed vector).

Preferably, the moving reference frame (Rₘ) is selected so that one of its axes is colinear with the forearm 22 when the measuring device 20 is worn. Here and hereinafter, the moving reference frame (Rₘ) is defined as being a direct orthogonal reference frame formed by a triplet of axes, shown in FIG. 2, comprising:
an axis U collinear with the forearm 22 and oriented towards the hand 29,
an axis V orthogonal to the axis U, and
an axis W orthogonal to the axes U and V.

The inertial device 33 also comprises an accelerometer 42 for measuring an acceleration of the measuring device 20 according to a system of three orthogonal axes defining a moving reference frame integral with the measuring device 20, which is advantageously the same as the moving reference frame (Rₘ) of the gyrometer 40. In other words, the accelerometer is capable of measuring the three components of an acceleration vector in said moving reference frame. It is thus understood that the accelerometer 42 is typically made up of a set of three accelerometers each associated with one of the three axes, in particular in tri-axis (that is to say each capable of measuring one of the three components of the acceleration vector). These accelerometers are sensitive to external forces, including gravitational forces, applied to the inertial device 33, and allow to measure a specific acceleration.

The data processing unit 34 is configured to deduce from the measurements of the inertial device 33 an orientation, a linear speed and a position of a measurement point integral with the measuring device 20, for example constituted by the origin of the moving reference frame (Rₘ) of the gyrometer 40 and the accelerometer 42. It is also configured to estimate a linear speed and a position of the center of gravity of the forearm 22. It is further configured to evaluate and analyze the forces of the muscles of the upper limb 12.

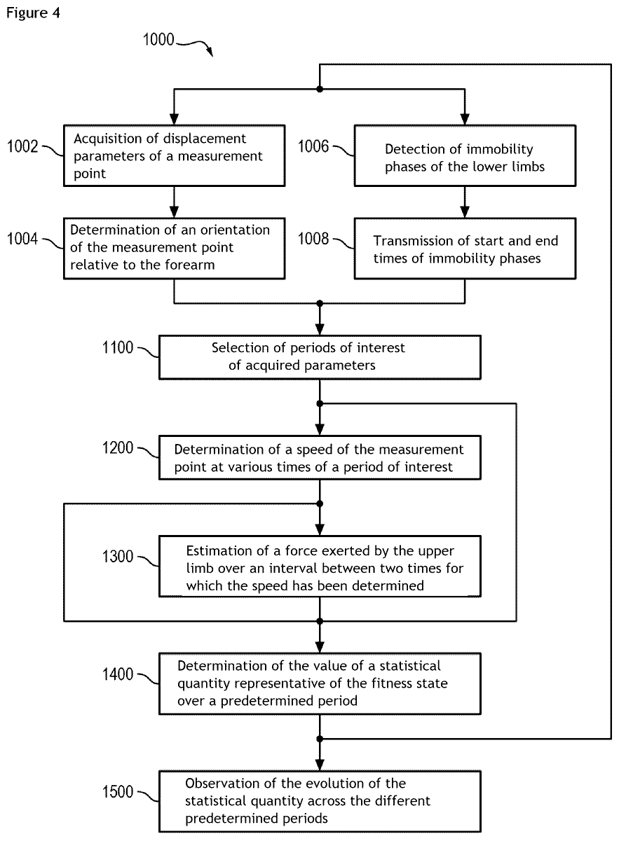

For this purpose, the data processing unit 34 is, in the example shown, constituted by a programmable machine, such as a DSP (“Digital Signal Processor”) or a microcontroller. It comprises a processor or CPU (“Central Processing Unit”) 44 and a memory 46 of the RAM (“Random Access Memory”) and/or ROM (“Read Only Memory”) type. The processor 44 is configured to execute instructions loaded into the memory 46. When the measuring device 20 is powered up, the processor 44 is capable of reading instructions from the memory 46 and executing them. These instructions form a computer program causing the implementation, by the processor 44, of certain steps of a method 1000 (FIG. 4) which will be detailed below.

Alternatively (not shown), the data processing unit 34 is constituted by a machine or a dedicated component, such as an FPGA (“Field-Programmable Gate Array”) or an ASIC (“Application-Specific Integrated Circuit”).

The data processing unit 34 moreover comprises a buffer memory 48 for the temporary storage of information necessary for the implementation of the method 1000.

The communication system 36 is configured to implement short-range wireless communication, for example Bluetooth or Wi-Fi (in particular in the example shown with the movement sensor 24) and/or to connect to a mobile network (typically UMTS/LTE/5G) for long-distance communication. Alternatively (not shown), the communication system 36 is for example a wired connection (typically USB) to transfer data from the storage module 38 to another storage module, typically a server (not shown).

For example, the communication system 36 is configured to receive from the movement sensor 24 displacement data of at least one lower limb of the individual 14 and/or to transmit the analysis data to an external device for presentation to a human operator, typically for display on a screen.

Optionally, the measuring device 20 also comprises an array of magnetometers (not shown) connected to the support 31, that is to say that they each have a movement substantially identical to that of the support 31 in the terrestrial reference frame, and spatially spaced from each other. Each magnetometer is a three-axis magnetometer capable of measuring a magnetic field along three axes. For this purpose, each magnetometer is typically constituted by three single-axis magnetometers (not shown) oriented along axes substantially perpendicular to each other. These axes are preferably the same as those of the system of three orthogonal axes of the gyrometer 40 and/or the accelerometer 42.

The array of magnetometers is suitable, due to its particular geometry, for allowing the determination, at each measurement time of the magnetometers, of a spatial gradient of the measured magnetic field, in particular of the coefficients of this gradient along each of the axes U, V, W of the moving reference frame (Rₘ) connected to the support 31. Each coefficient of said gradient is for example determined by a method using the vector measurements of the magnetic field carried out by the magnetometers, associated with optimization methods of the least square or median filter type or associated with the intrinsic properties of the magnetic field described by Maxwell's equations. However, any other conventional method adapted to calculate the coefficients of the spatial gradient of the magnetic field is suitable.

The data processing unit 34 is then typically configured to determine the linear speed of the measurement point, over certain displacement phases, by implementing the method described in EP 2 541 199.

In the example described above, the processing unit 34 of the measuring device 20 is mounted on the support 31. Alternatively (not shown), at least part of the data processing unit 34 is remote, for example in a mobile terminal (not shown) and/or in a remote server (not shown). In other words, at least part of the steps of the method 1000 is carried out by a mobile terminal and/or a remote server. The communication system 36 is then configured to send to the mobile terminal and/or to the remote server the data from the inertial device 33 and, where appropriate, from the magnetometer array of the first measuring device 20.

Returning to FIG. 1, the movement sensor 24 is here secured to a lower limb 50 of the individual 14.

It is recalled here that, anatomically, a lower limb such as the lower limb 50 is made up of three segments: the thigh 51, articulated to the torso 26 by the hip 52, the leg 53, articulated to the thigh 51 by the knee 54, and the foot 55, articulated to the leg 53 by the ankle 56. Here and hereinafter, it is in this sense that these terms are used, the term “leg” being in particular used to designate only the segment of the upper limb between the knee and the ankle and not, as in common language, the entire lower limb.

The movement sensor 24 is in particular secured to the leg 53 of the lower limb 50, that is to say that it has, in the terrestrial reference frame, a movement substantially identical to that of the leg 53. It is in particular placed between the knee 54 (excluded) and the ankle 56 (included), for example, as shown, substantially on the ankle 56.

Alternatively, the movement sensor 24 is fastened to a wheelchair 58 in which the individual 14 is seated.

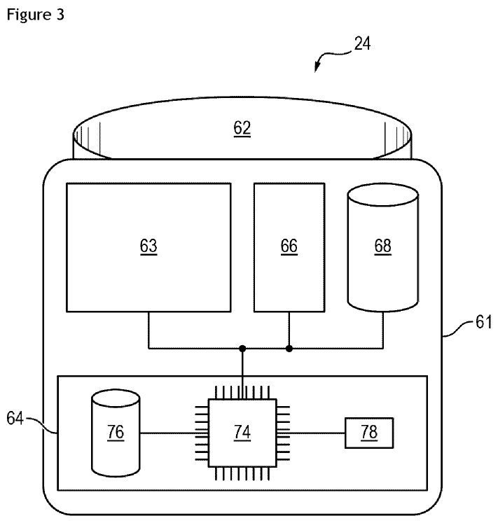

With reference to FIG. 3, the movement sensor 24 here comprises a support 61 and an attachment element 62 for attaching the support 61 to the leg 53. It also comprises a gyrometer 63 and a data processing unit 64 mounted on the support 61. In the example shown, it further comprises a communication system 66, typically a wireless communication system, mounted on the support 61 for communication of the movement sensor 24 with the measuring device 20 and/or an external device such as a mobile terminal (not shown), for example a multifunction mobile, or a remote server (not shown). Optionally, it further comprises a storage module 68 mounted on the support 61.

The support 61 is typically constituted by a casing.

The attachment element 62 is here constituted by a bracelet, for example with hook-and-loop strip, adapted to enclose the leg 53 and allow the integral connection. Alternatively (not shown), the attachment element 62 is constituted by any element allowing an integral connection with the leg 53.

The gyrometer 63 is capable of measuring an angular speed of the movement sensor 24 according to a system of three orthogonal axes defining a moving reference frame integral with the movement sensor 24, that is to say measuring the three components of an angular speed vector in said moving reference frame. It is thus understood that the gyrometer 63 is typically constituted by a set of three gyrometers each associated with one of the three axes, in particular in tri-axis (that is to say each capable of measuring one of the three components of the angular speed vector).

The data processing unit 64 is configured to deduce from the measurements of the gyrometer 63 the movement phases and the immobility phases of the movement sensor 24. For this purpose, the data processing unit 64 is typically configured to compare the norm of the angular speed measured by the gyrometer 63 with a threshold, and to define the phases during which this norm is less than the threshold as being immobility phases and the phases during which this norm is less than the threshold as being movement phases.

The data processing unit 64 is, in the example shown, constituted by a programmable machine, such as a DSP (Digital Signal Processor) or a microcontroller. It comprises a processor or CPU (Central Processing Unit) 74 and a memory 76 of the RAM (Random Access Memory) and/or ROM (Read Only Memory) type. The processor 74 is configured to execute instructions loaded into the memory 76. When the movement sensor 24 is powered up, the processor 74 is capable of reading instructions from the memory 76 and executing them. These instructions form a computer program causing the processor 74 to implement a method for detecting a displacement of the movement sensor 24.

Alternatively (not shown), the data processing unit 64 is constituted by a machine or a dedicated component, such as an FPGA (“Field-Programmable Gate Array”) or an ASIC (“Application-Specific Integrated Circuit”).

The data processing unit 64 moreover comprises a buffer memory 78 for the temporary storage of information necessary for detecting the displacement of the movement sensor 24.

The communication system 66 is configured to implement short-range wireless communication, for example Bluetooth or Wi-Fi (in particular in the example shown with the measuring device 20) and/or to connect to a mobile network (typically UMTS/LTE/5G) for long-distance communication. Alternatively (not shown), the communication system 66 is for example a wired connection (typically USB) to transfer data from the storage module 68 to another storage module, typically a server (not shown).

For example, the communication system 66 is configured to transmit the start and end times of the immobility phases to the measuring device 20 and/or to an external device, typically a mobile telephone or a server.

A method 1000 implemented by the equipment 10 will now be described, with reference to FIGS. 4 to 8.

As can be seen in FIG. 4, the method 1000 begins with a step 1002 of acquiring displacement parameters of the measurement point. During this step, the inertial device 33 acquires the acceleration \vec{y}(tₖ) and the rotation speed \vec{ω}(tₖ) of the measurement point at each of a plurality of sampling times tₖ. It will be noted that the acceleration \vec{γ}(tₖ) and the rotation speed \vec{ω}(tₖ) are here vectors each having three components along each of the axes U, V, W of the moving reference frame (Rₘ) attached to the support 31 of the measuring device 20. The sampling times tₖ are spaced from each other by a time dt that is very small compared to the time characteristic of the movements of the individual 14, this time dt being for example substantially equal to 10 ms.

Step 1002 follows a start-up of the measuring device 20 and typically lasts until a shutdown of the measuring device 20.

The method 1000 further comprises, after step 1002 or, more precisely, after the acquisition of the acceleration \vec{γ}(tₖ) and rotation speed \vec{ω}(tₖ) vectors for a sufficient number of sampling times tₖ, a step 1004 of determining an orientation of the measurement point relative to the forearm 22. This step allows to know the orientation of the moving reference frame (Rₘ) attached to the support 31 of the measuring device 20 relative to the forearm 22. Indeed, if the orientation of said moving reference frame (Rₘ) relative to the support 31 is known, two positions of the support 31 relative to the forearm 22 are possible, depending on the direction in which the attachment element 32 is mounted on the forearm 22: a position in which the axis U of the moving reference frame (Rₘ) colinear with the forearm 22 points towards the hand 29 and a position in which said axis points towards the elbow 28.

For example, step 1004 comprises the detection, by the processing unit 34, of immobility phases of the measuring device 20, followed by the measurement of the acceleration during said immobility phases. The detection of the immobility phases is typically carried out by comparing the norm of the rotation speed \vec{ω}(tₖ) with a threshold, an immobility phase being detected when, for several successive sampling times tₖ, the norm of the rotation speed \vec{ω}(tₖ) is less than said threshold. The measurement of the acceleration during these immobility phases gives the orientation of the gravitational acceleration in the moving reference frame (Rₘ), from which the processing unit 34 deduces the orientation of the measurement point relative to the forearm 22.

If it follows from this deduction that the axis U points towards the hand 29, then the moving reference frame (Rₘ) is maintained. If, on the contrary, the axis U points towards the elbow 28, then the moving reference frame (Rₘ) is reoriented (it is typically rotated 180° around its axis V or its axis W) so that the axis U points towards the hand 29.

In parallel with steps 1002 and 1004, the method 1000 also comprises a step 1006 of detecting phases of immobility of the lower limbs of the individual 14, followed by a step 1008 of transmitting the start and end times of these phases of immobility to the measuring device 20.

In step 1006, the processing unit 64 of the movement sensor 24 typically compares the rotation speed standard measured by the gyrometer 63 with a threshold, and determines the immobility phases as being the periods during which said standard is less than the threshold.

Then, in step 1008, the movement sensor 24 transmits to the measuring device 20, via the communication systems 66, 36, the start and end times of the immobility phases thus detected.

Steps 1004 and 1008 are followed by a step 1100 of selecting periods of interest for the acquired parameters. During this step 1100, the processing unit 34 of the measuring device 20 identifies the periods between the start and end times of the immobility phases of the lower limbs as forming periods of interest for the acquired parameters and selects these periods. The accelerations \vec{γ}(tₖ) and rotation speeds \vec{ω}(tₖ) acquired outside of these periods are then discarded.

Then, for each period of interest, the method 1000 comprises a step 1200 of determining a linear speed of the measurement point at various times of the period of interest. During this step 1200, the processing unit 34 determines the linear speed of the measurement point during certain phases.

Said phases are for example phases of immobility of the measuring device 20 that the processing unit 34 detects and for which it determines a zero linear speed of the measurement point during these phases. These immobility phases are typically detected by comparing the rotation speed norm \vec{ω}(tₖ) with a threshold, an immobility phase being detected when, for several successive sampling times tₖ, the rotation speed norm \vec{ω}(tₖ) is less than said threshold.

Preferably, said phases also include phases of immobility of the elbow 28, which the processing unit 34 detects when the movement of the measurement point is a pure rotation of radius equal to the distance between the measurement point and the elbow 28. The linear speed of the measurement point is then easily determined, in a manner known to the person skilled in the art.

Advantageously, said phases also include phases during which the linear speed of the measurement point is determined by means of data other than that provided by the inertial device 33. These other data consist, for example, of magnetic field gradient measurement data provided by the magnetometer array. The phases during which the linear speed of the measurement point is determined then include phases of stationarity of the ambient magnetic field, the processing unit 34 then implementing the method described in WO 2019/016474 to determine the linear speed of the measurement point during these phases.

The phases during which the linear speed of the measurement point is determined are spaced from each other by time intervals during which the linear speed of the measurement point is not a priori known. The method 1000 comprises, for each of these intervals, a step 1300 of estimating a force exerted by the muscles of the upper limb during this interval.

This step 1300 is implemented by the processing unit 34.

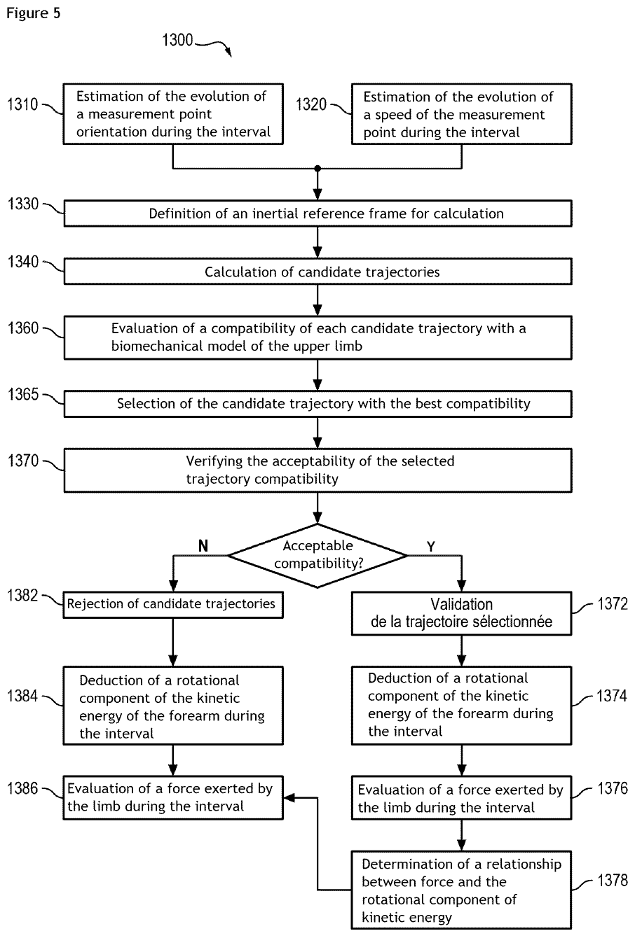

With reference to FIG. 5, step 1300 comprises a first sub-step 1310 of estimating the evolution of an orientation of the measurement point during the interval. “Orientation of the measurement point”, here and hereinafter means an orientation of the moving reference frame (Rₘ) in a terrestrial inertial reference frame (Rᵢ) having an axis coincident with the vertical, such that the angular speed \vec{ω}(tₖ) is equal to the rotation speed of the moving reference frame (Rₘ) relative to said inertial reference frame (Rᵢ). Here and hereinafter, the inertial reference frame (Rᵢ) is defined as being a direct orthogonal reference frame formed of a triplet of axes, shown in FIG. 1, comprising:
an axis Z coincident with the vertical,
a horizontal axis X, and
a horizontal axis Y forming with the axes X and Z a direct orthogonal reference frame X, Y, Z.

The orientation of the measurement point can be given by a rotation matrix (denoted R), an orientation quaternion (denoted Q), or Euler angles (roll φ, pitch θ, yaw ψ). These three notations being equivalent, they are used interchangeably in this document.

It is known in particular that the formula giving the matrix of the transition from the inertial reference frame (Rᵢ) to the moving reference frame (Rₘ) from the Euler angles is written:

$${R}_{{R}_{i} \to {R}_{m}} = [ \begin{matrix}cos θ \cdot cos ψ & - cos θ \cdot sin ψ & sin θ \\ \begin{matrix}cos ψ \cdot sin θ \cdot sin φ + \\ cos φ \cdot sin ψ\end{matrix} & \begin{matrix}cos φ \cdot cos ψ - sin θ + \\ sin φ \cdot sin ψ\end{matrix} & - cos θ \cdot sin φ \\ \begin{matrix}sin φ \cdot sin ψ - cos φ \cdot \\ cos ψ \cdot sin θ\end{matrix} & \begin{matrix}cos ψ \cdot sin φ + cos φ \cdot \\ sin θ \cdot sin ψ\end{matrix} & cos θ \cdot cos φ\end{matrix} ]$$

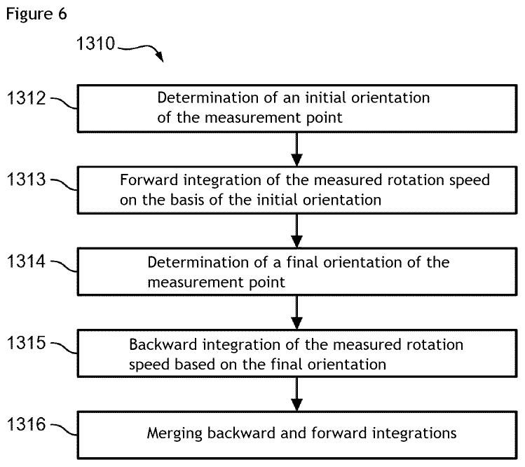

With reference to FIG. 6, sub-step 1310 comprises a first sub-step 1312 of determining an initial orientation of the measurement point at an initial time tᵢ of the start of the interval.

In this sub-step 1312, the processing unit 34 first determines a starting orientation of the measurement point at a starting time t₀ at which the forearm 22 and therefore the measuring device 20 is considered immobile. This determination is typically carried out from the acceleration measurements, the measured acceleration \vec{γ}(t₀) being equal to the opposite of the gravitational field: \vec{γ}(t₀)=−\vec{g}. The rotation matrix at the starting time t₀ is then such that it solves the following equation:

$$R ( {t}_{0} ) \vec{Z} = \frac{\vec{γ} ( {t}_{0} )}{‖ \vec{γ} ( {t}_{0} ) ‖}$$

in which:
   Z is the vertical unit vector of the inertial reference frame (Rᵢ),
   \vec{γ}(t₀) is the acceleration measured by the accelerometer 42 at the starting time
   t₀, expressed in the moving reference frame (Rₘ), and
   ∥\vec{γ}(t₀)∥ is the norm of the acceleration measured \vec{γ}(t₀) at time t₀.

Note that this equation is not fully deterministic and leaves degrees of freedom on some components of the rotation matrix R(t₀). This is because the yaw angle ψ(t₀) at the starting time t₀ is of little importance for the rest of the calculations and the axis X can therefore be set arbitrarily. For example, the axis X is selected so that the yaw angle ψ(t₀) at the starting time t₀ is zero, that is to say so that the rotation matrix at the starting time t₀ solves the following additional equation:

$$R ( {t}_{0} ) \vec{Y} = \frac{\vec{γ} ( {t}_{0} )}{‖ \vec{γ} ( {t}_{0} ) ‖} ∧ \vec{U}$$

in which:
   \vec{γ} is the unit vector of the inertial reference frame (Rᵢ) corresponding to the axis γ,
   \vec{U} is the unit vector of the moving reference frame (Rₘ) corresponding to the axis U,
   \vec{γ}(t₀) is the acceleration measured by the accelerometer 42 at the starting time t₀, expressed in the moving reference frame (Rₘ),
   ∥\vec{γ}(t₀)∥ is the norm of the acceleration measured \vec{γ}(t₀) at time t₀, and
   ∧ A is the vector product operator.

Preferably, the starting time t₀ is equal to the initial time tᵢ; the initial orientation is then equal to the starting orientation. Alternatively, the starting time t₀ is prior to the initial time tᵢ, the initial orientation then being deduced from the starting orientation by forward integration of the measurements provided by the gyrometer 40. “Forward integration” means here and hereinafter that the integration is carried out in positive time, with an initial value chosen in the past.

Sub-step 1312 is followed by a sub-step 1313 of forward integration of the measured orientation on the basis of the initial orientation.

During this sub-step 1313, the processing unit 34 produces a first estimate Rf of the orientation of the measurement point at each sampling time tₖ of the interval by integrating the rotation speed measurements prior to the time tₖ, taking as initial value the initial orientation at the time tᵢ.

Preferably, this determination comprises the implementation of a linear (Luenberger filter, Kalman filter, etc.) or non-linear (extended Kalman filter, invariant observer, etc.) state estimator filter. In the present description, the implementation of an extended Kalman filter is described, but the person skilled in the art will be able to transpose to other filters.

The implementation of said extended Kalman filter firstly comprises a prediction step, during which the processing unit 34 uses the following formula:

R_{f}(tₖ)=R_{f}(tₖ₋₁)+\dot{R}_{f}(tₖ)dt

in which tₖ₋₁ is the sampling time immediately preceding the sampling time tₖ, and \dot{R}_{f}(tₖ) is the temporal derivative of the rotation matrix of the moving reference frame (Rₘ) in the inertial reference frame (Rᵢ), given by the following differential equation:

$${\dot{R}}_{f} ( {t}_{k} ) = [ \begin{matrix}0 & - {ω}_{z} ( {t}_{k} ) & {ω}_{y} ( {t}_{k} ) \\ {ω}_{z} ( {t}_{k} ) & 0 & - {ω}_{z} ( {t}_{k} ) \\ - {ω}_{y} ( {t}_{k} ) & {ω}_{x} ( {t}_{k} ) & 0\end{matrix} ] \times {R}_{f} ( {t}_{k - 1} )$$

Noting \hat{R}ₙ the matrix estimate R_{f} after n sample steps, the matrix estimate \hat{R}ₙ for each measurement \vec{ωₙ} is:

$${\hat{R}}_{n + 1} = {\hat{R}}_{n} + [ \begin{matrix}0 & - {ω}_{n , z} & {ω}_{n , y} \\ {ω}_{n , z} & 0 & - {ω}_{n , x} \\ - {ω}_{n , y} & {ω}_{n , x} & 0\end{matrix} ] \times {\hat{R}}_{n} dt$$

The equation is given here for a rotation matrix but an equivalent differential equation exists between attitude quaternion and angular speed.

In parallel, the processing unit 34 modifies, by linearization of the differential equation, a covariance matrix estimating the covariance between each state of the filter.

The prediction step is followed by an update step.

This update step uses the fact that the acceleration y measured by the accelerometer 42 is equal to the sum of the gravity field g and the acceleration a related to the displacements of the measuring device 20:

\vec{γ}=\vec{a}+\vec{g}

In addition, it is known that the acceleration \vec{a} is on average zero (it is understood that the upper limb 12 regularly returns to its starting position and that the movements in one direction or the other statistically compensate for each other). It is deduced that the measured acceleration \vec{γ} expressed in the terrestrial inertial reference frame (Rᵢ) is equal on average to −\vec{g}.

From the rotation matrix \hat{R}ₙ and the accelerometer measurements in the moving reference frame (Rₘ), it is possible to express the acceleration measured \vec{γ} in the inertial reference frame (Rᵢ):

$${\hat{R}}_{n}^{t} [ \begin{matrix}{γ}_{n , x} \\ {γ}_{n , y} \\ {γ}_{n , z}\end{matrix} ]$$

The difference between the estimated gravity field and the actual Earth gravity field is then written as:

$${error}_{n} = {\hat{R}}_{n}^{t} [ \begin{matrix}{γ}_{n , x} \\ {γ}_{n , y} \\ {γ}_{n , z}\end{matrix} ] - [ \begin{matrix}0 \\ 0 \\ g\end{matrix} ]$$

This difference, which must be zero on average, is used for orientation recalibration in the update step.

Moreover, by linearizing this formula, it is possible to calculate the Kalman gain and consequently update the state and the covariance matrix. The update of the covariance matrix is based on the assumption that the errors due to the sensors and the approximations are modeled as Gaussian distribution noise. The variance is estimated by measuring the noise of the sensors at rest and from assumptions on the movements made by the forearm 22.

Thus a first estimate of the orientation of the measurement point at each sampling time tₖ of the interval is obtained.

Sub-step 1313 is followed by a sub-step 1314 of determining a final orientation of the measurement point at a final time tr at the end of the interval.

In this sub-step 1314, the processing unit 34 first determines a reference orientation of the measurement point at a reference time tᵣ at which the forearm 22 and therefore the measuring device 20 is considered immobile. This determination is typically carried out from the acceleration measurements, the measured acceleration γ(tᵣ) being equal to the opposite of the gravitational field: \vec{γ}(tᵣ)=−\vec{g}. The rotation matrix at the reference time tᵣ is then such that it solves the following equation:

$$R ( {t}_{r} ) \vec{Z} = \frac{\vec{γ} ( {t}_{r} )}{‖ \vec{γ} ( {t}_{r} ) ‖}$$

in which:
   \vec{Z} is the vertical unit vector of the inertial reference frame (Rᵢ),
   \vec{γ}(tᵣ) is the acceleration measured by the accelerometer 42 at the reference time
   tᵣ, expressed in the moving reference frame (Rₘ), and
   ∥\vec{γ}(tᵣ)∥ is the norm of the acceleration measured \vec{γ}(tᵣ) at time tᵣ.

This equation allows to obtain the roll and pitch at the reference time tᵣ, which are given by the following formulas:

$$φ ( {t}_{r} ) = - {tan}^{- 1} \frac{{γ}_{v} ( {t}_{r} )}{{γ}_{w} ( {t}_{r} )}$$

$$θ ( {t}_{r} ) = {sin}^{- 1} \frac{{γ}_{u} ( {t}_{r} )}{\sqrt{{{γ}_{u} ( {t}_{r} )}^{2} + {{γ}_{v} ( {t}_{r} )}^{2} + {{γ}_{w} ( {t}_{r} )}^{2}}}$$

in which γᵤ is the component of the measured acceleration \vec{γ} along the axis U, γᵥ is the component of the measured acceleration \vec{γ} along the axis V and γ_{w} is the component of the measured acceleration \vec{γ} along the axis W.

The yaw angle ψ(tᵣ) is set so as to be equal to the yaw angle obtained by forward integration of the rotation speed measurements from the starting time t₀ to the reference time tᵣ.

Preferably, the reference time tᵣ is equal to the final time t_{f}; the final orientation is then equal to the reference orientation. Alternatively, the reference time tᵣ is later than the final time tr, the final orientation then being deduced from the reference orientation by backward integration of the measurements provided by the gyrometer 40. “Backward integration” means here and hereinafter that the integration is carried out in negative time, with an initial value chosen in the future.

Sub-step 1314 is followed by a sub-step 1315 of backward integration of the measured orientation based on the final orientation.

During this sub-step 1315, the processing unit 34 produces a second estimate Rb of the orientation of the measurement point at each sampling time tₖ of the interval by integrating the rotation speed measurements subsequent to the time tₖ, taking as initial value the final orientation at the time t_{f}.

Preferably, this determination comprises the implementation of a linear state estimator filter (Luenberger filter, Kalman filter, etc.) or non-linear (extended Kalman filter, invariant observer, etc.) state estimator filter. The implementation of an extended Kalman filter is described in the present description, but the person skilled in the art will be able to transpose to other filters.

This implementation is substantially identical to that of the forward integration step 1313 described above, with the following differences:
the formula used in the prediction step is:

R_{b}(tₖ)=R_{b}(tₖ₊₁)−\dot{R}_{b}(tₖ)dt
where tₖ₊₁ is the sampling time immediately following the sampling time tₖ, and
the differential equation giving the instantaneous rotation matrix \dot{R}_{b}(tₖ) of the moving reference frame (Rₘ) in the inertial reference frame (Rᵢ) is expressed as follows:

$${R}_{b} ( {t}_{k} ) = [ \begin{matrix}0 & - {ω}_{z} ( {t}_{k} ) & {ω}_{y} ( {t}_{k} ) \\ {ω}_{z} ( {t}_{k} ) & 0 & - {ω}_{x} ( {t}_{k} ) \\ - {ω}_{y} ( {t}_{k} ) & {ω}_{x} ( {t}_{k} ) & 0\end{matrix} ] \times {R}_{b} ( {t}_{k + 1} )$$

In essence, these differences can be summarized as follows: while forward integration uses prior estimates for the prediction step of the extended Kalman filter, backward integration uses posterior estimates.

Thus a second estimate of the orientation of the measurement point at each sampling time tₖ of the interval is obtained.

Sub-step 1310 concludes with a sub-step 1316 of merging the backward and forward integrations, during which a final estimate of the orientation of the measurement point at each sampling time tₖ of the interval is calculated by linear-spherical interpolation of the first and second estimates:

R(tₖ)=Slerp(R_{f}(tₖ),R_{b}(tₖ),i)
where:
   R_{f}(tₖ) is the first estimate of the orientation obtained in sub-step 1313 by forward integration,
   R_{b}(tₖ) is the second estimate of the orientation obtained in sub-step 1315 by backward integration, and
   i is given by the following formula:

$$i = \frac{{t}_{k} - {t}_{i}}{{t}_{f} - {t}_{k}}$$

Returning to FIG. 5, step 1300 comprises, in parallel with step 1310, a step 1320 of estimating a linear speed of the measurement point during the interval.

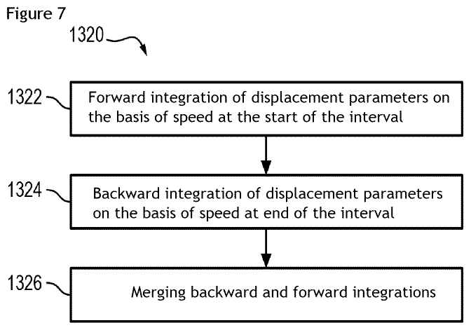

With reference to FIG. 7, this sub-step 1320 comprises a first sub-step 1322 of forward integration of the displacement parameters on the basis of the linear speed at the start of the interval.

During this sub-step 1322, the processing unit 34 produces a first estimate V_{f} of the linear speed of the measurement point at each sampling time tₖ of the interval by integrating the acceleration and rotation speed measurements prior to time tₖ, taking as initial value an initial speed at time tᵢ. This initial speed is equal to the linear speed of the measurement point at the end of a phase for which the linear speed was determined in step 1200 and which immediately precedes the interval.

The first estimate of the linear speed of the measurement point is estimated iteratively at each sampling time tₖ, starting from the oldest, using the following formula:

\vec{V_{f}}(tₖ)=\vec{V_{f}}(tₖ₋₁)+\vec{\dot{V}_{f}}(tₖ)dt

Where \vec{V_{f}}(tₖ) is the first estimate of the linear speed at sampling time tₖ, \vec{V_{f}}(tₖ₋₁) is the first estimate of the linear speed at a sampling time tₖ₋₁ immediately preceding the sampling time tₖ (and therefore estimated at the previous iteration), and \vec{\dot{V}_{f}}(tₖ) is the acceleration at the sampling time tₖ, given by the following formula:

\vec{\dot{V}_{f}}(tₖ)=\vec{γ}(tₖ)+R_{R}_{ᵢ}_{-R}_{ₘ}(tₖ)\vec{g}−\vec{ω}(tₖ₋₁)∧\vec{V_{f}}(tₖ₋₁)
where:
   \vec{γ}(tₖ) is the acceleration measured by the accelerometer 42 at time tₖ, expressed in the moving reference frame (Rₘ),
   R_{R}_{ᵢ}_{→R}_{ₘ}(tₖ) is the rotation matrix giving the orientation of the measurement point in the inertial reference frame, said rotation matrix being either equal to the first estimate R_{b}(tₖ) of the orientation at the sampling time tₖ estimated during the sub-step 1313, or equal to the final estimate R(tₖ) of the orientation at the sampling time tₖ estimated during the sub-step 1316,
   \vec{g} is the gravitational field expressed in the inertial reference frame (Rᵢ),
   \vec{ω}(tₖ₋₁) is the rotation speed measured by the gyrometer 40 at time tₖ₋₁, expressed in the moving reference frame (Rₘ), and
   ∧ is the vector product operator.

This gives a first estimate of the linear speed of the measurement point during the interval.

Sub-step 1322 is followed by a sub-step 1324 of backward integration of the displacement parameters based on the linear speed at the end of the interval.

During this sub-step 1324, the processing unit 34 produces a second estimate V_{b} of the linear speed of the measurement point at each sampling time tₖ of the interval by integrating the acceleration and rotation speed measurements subsequent to the time tₖ, taking as an initial value a final speed at the time tr. This final speed is equal to the linear speed of the measurement point at the start of a phase for which the linear speed was determined in step 1200 and which immediately follows the interval.

The second estimate of the linear speed of the measurement point is estimated iteratively at each sampling time tₖ, starting from the most recent, using the following formula:

\vec{V_{b}}(tₖ)=\vec{V_{b}}(tₖ₊₁)−\vec{\dot{V}_{b}}(tₖ)dt
in which \vec{V_{b}}(tₖ) is the second estimate of the linear speed at the sampling time tₖ, \vec{V_{b}}(tₖ₊₁) is the second estimate of the linear speed at a sampling time tₖ₊₁ immediately following the sampling time tₖ (and therefore estimated at the previous iteration), and \vec{\dot{V}_{f}}(tₖ) is the acceleration at the sampling time tₖ, given by the following formula:

\vec{\dot{V}_{f}}(tₖ)=\vec{γ}(tₖ)+R_{R}_{ᵢ}_{-R}_{ₘ}(tₖ)\vec{g}−\vec{ω}(tₖ₊₁)∧\vec{V_{b}}(tₖ₊₁)
where:
   \vec{γ}(tₖ) is the acceleration measured by the accelerometer 42 at time tₖ, expressed in the moving reference frame (Rₘ),
   R_{R}_{ᵢ}_{→R}_{ₘ}(tₖ) is the rotation matrix giving the orientation of the measurement point in the inertial reference frame, said rotation matrix being either equal to the second estimate R_{b}(tₖ) of the orientation at the sampling time tₖ estimated in sub-step 1315, or equal to the final estimate R(tₖ) of the orientation at the sampling time tₖ estimated in sub-step 1316,
   \vec{g} is the gravitational field expressed in the inertial reference frame (Rᵢ), and
   \vec{ω}(tₖ₊₁) is the rotation speed measured by the gyrometer 40 at time tₖ₊₁, expressed in the moving reference frame (Rₘ), and
   ∧ is the vector product operator.

This gives a second estimate of the linear speed of the measurement point during the interval.

Sub-step 1320 concludes with a sub-step 1326 of merging the backward and forward integrations, during which a final estimate of the linear speed of the measurement point at each sampling time tₖ of the interval is calculated by linear interpolation of the first and second estimates:

\vec{V}(tₖ)=i\vec{V_{b}}(tₖ)+(1−i)\vec{V_{f}}(tₖ)
where:
   \vec{V_{f}}(tₖ) is the first estimate of the linear speed obtained in sub-step 1322 by forward integration,
   \vec{V_{b}}(tₖ) is the second estimate of the linear speed obtained in sub-step 1324 by backward integration, and
   i is given by the following formula:

$$i = \frac{{t}_{k} - {t}_{i}}{{t}_{f} - {t}_{k}} .$$

This gives an estimate of the change in orientation and an estimate of the change in linear speed of the measurement point during the interval.

Returning to FIG. 5, sub-steps 1310 and 1320 are followed by a sub-step 1330 of defining a calculation inertial reference frame.

This calculation inertial reference frame is for example the same as the inertial reference frame (Rᵢ) used for estimating the orientation and the linear speed. Alternatively, this calculation inertial reference frame is any other inertial reference frame and preferably a reference frame having:
a first axis Z coincident with the vertical,
a second horizontal axis X, oriented in the direction in which the axis of the moving reference frame (Rₘ) collinear with the forearm 22 pointed when said axis had the most horizontal orientation of the interval, and
a third horizontal axis Y forming with the first and second axes a direct orthogonal reference frame X, Y, Z.

The orientations R(tₖ) and the previously estimated speeds \vec{V}(tₖ) are then recalculated to be expressed in said inertial calculation reference frame, by means of a simple change of reference frame well known to the person skilled in the art.

Sub-step 1330 is in turn followed by a sub-step 1340 of calculating candidate trajectories for the center of gravity of the forearm 22.

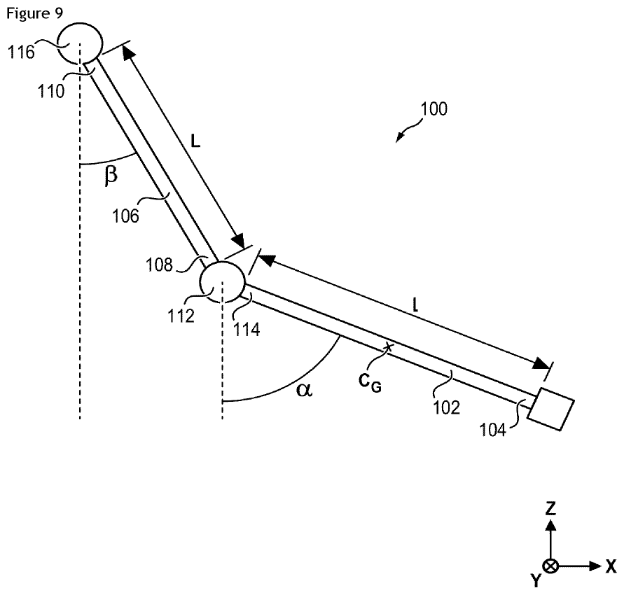

This calculation is based on a biomechanical model 100 of the upper limb 12, shown in FIG. 9.

As visible in this Figure, the biomechanical model 100 comprises a first segment 102 having a free end 104. It also comprises a second segment 106 articulated by a first 108 of its ends 108, 110, via a first ball joint 112, to a proximal end 114 of the first segment 102 opposite the free end 104, and articulated by its second end 110, via a second ball joint 1116, to a fixed point.

The first segment 102 models the forearm 22 of the upper limb 12. It is constituted by a cylinder having a radius r and a length I equal to the distance between the measuring device 20 and the elbow 28. It forms with the vertical Z a first primary angle α modeling an angle of the forearm 22 with the vertical, and with the plane (X, Z) a first secondary angle ζ (not shown) modeling an angle of the forearm 22 with said plane.

The second segment 106 models the arm 25 of the upper limb 12. It is constituted by a cylinder having a length L equal to the length of said arm 25. It forms with the vertical a second primary angle β modeling an angle of the arm 25 with the vertical, and with the plane (X, Z) a second secondary angle n (not shown) modeling an angle of the arm 25 with said plane.

Each of the segments 102, 106 is rigid, that is to say it is not bendable. Preferably, at least one of the segments 102, 106 is extensible. If both segments 102, 106 are extensible, these extensions are advantageously constrained so as to be equal to each other.

The center of gravity CG of the forearm 22 is placed on the first segment 102, at a distance from the joint 112 substantially equal to half the length of the forearm 22.

The biomechanical model 100 thus has a plurality of possible configurations, each defined by the values of the first primary α and secondary ζ angles and the values of the second primary β and secondary η angles.

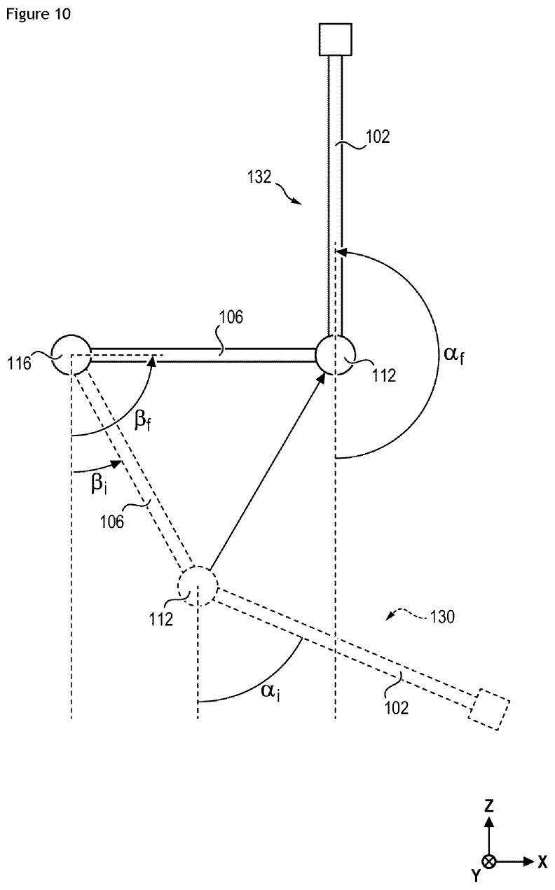

In sub-step 1340, a candidate trajectory is calculated for each of a plurality of pairs of starting and finishing configurations 130, 132 of the biomechanical model 100, shown in FIG. 10, respectively modeling the configuration of the upper limb 12 at the start and end of the interval. By hypothesis, the first secondary angle ζ is defined in each of these configurations as being equal to the heading y of the measurement point, and the second secondary angle η is set equal to the first secondary angle ζ. Each of the starting and finishing configurations of the biomechanical model 100 is therefore directly defined by the first and second primary angles α, β.

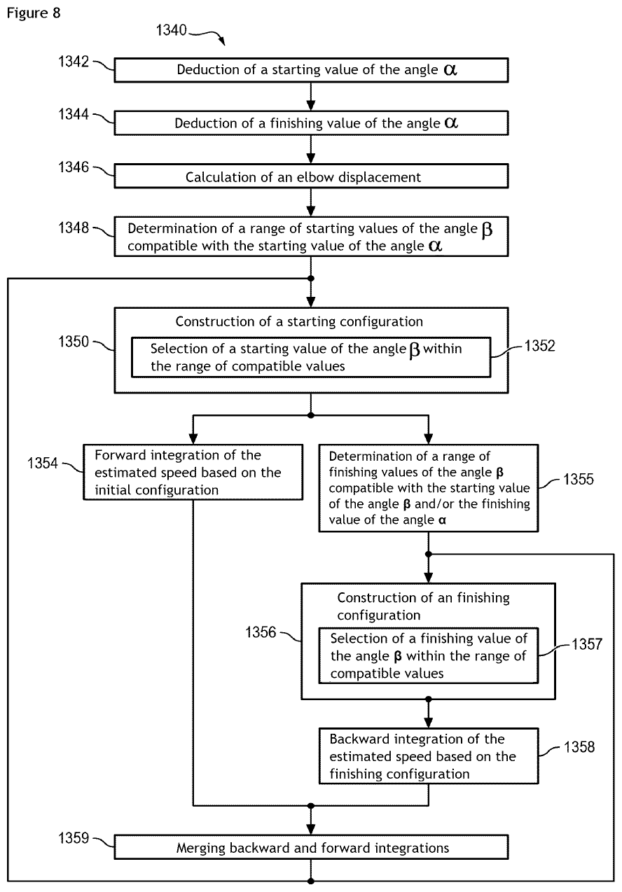

With reference to FIG. 8, sub-step 1340 thus begins, for the purposes of defining the starting and finishing configurations, with a sub-step 1342 of deducing a starting value αᵢ (FIG. 10) of the first primary angle α and a sub-step 1344 of deducing a finishing value α_{f} (FIG. 10) of the first primary angle α.

In sub-step 1342, the first primary starting angle αᵢ is deduced from the orientation of the measurement point at the initial time tᵢ, using the following formula:

αᵢ=arccos(\vec{Oᵢ}.\vec{Z})
in which \vec{Z} is the vertical unit vector of the inertial reference frame (Rᵢ) and \vec{Oᵢ} is the unit vector defining the direction of the forearm 22 in the inertial reference frame (Rᵢ) at the initial time tᵢ, said vector \vec{Oᵢ} being given by the following formula:

\vec{Oᵢ}=R^{t}(tᵢ)\vec{U}

where R^{t}(tᵢ) is the transpose of the rotation matrix at the initial time tᵢ and \vec{U} is the unit vector of the moving reference frame (Rₘ) corresponding to the axis U.

In sub-step 1344, the first primary finishing angle α_{f} is deduced from the orientation of the measurement point at the final time tr, using the following formula:

α_{f}=arccos(\vec{O_{f}}.\vec{Z})
in which \vec{Z} is the unit vector defined above and \vec{O_{f}} is the unit vector defining the direction of the forearm 22 in the inertial reference frame (Rᵢ) at the final time t_{f}, said vector \vec{O_{f}} being given by the following formula:

\vec{O_{f}}=R^{t}(t_{f})\vec{U}
where R^{t}(t_{f}) is the transpose of the rotation matrix at the final time t_{f} and \vec{U} is the unit vector of the moving reference frame (Rₘ) corresponding to the axis U.

These sub-steps 1342, 1344 are shown as being implemented one after the other. Alternatively, they are implemented in parallel to each other.

Sub-steps 1342, 1344 are followed by a sub-step 1346 of calculating a displacement of the elbow 28. Alternatively, they are implemented in parallel with this sub-step 1346.

In sub-step 1346, the processing unit 34 calculates a displacement \vec{Δ} of the elbow 28 between the initial time tᵢ and the final time t_{f}, from the speeds and orientations estimated during the sub-steps 1310, 1320. This displacement \vec{Δ} is given by the following formula:

$$\vec{Δ} = {\int }_{{t}_{i}}^{{t}_{f}} \vec{V ( t )} dt - l ( \vec{{O}_{f}} - \vec{{O}_{ι}} )$$

where \vec{V(t)} is the linear speed vector estimated at a time t of the interval and \vec{Oᵢ} and \vec{O_{f}} are the unit vectors defined above.

Sub-step 1340 also comprises, after sub-step 1342, a sub-step 1348 of determining a range of starting values of the second primary angle β compatible with the starting value di of the first primary angle α.

Preferably, this sub-step 1348 comprises:
the comparison of the displacement norm \vec{Δ} with the length L of the arm 25, and
the comparison with the value 1 of the difference between the cosine of the initial value αᵢ of the first primary angle α and the ratio of the norm of the displacement \vec{Δ} to the length L of the arm 25 (cos(αᵢ)−∥\vec{Δ}∥/L).

There are then different scenarios:
if the displacement norm \vec{Δ} is less than or equal to twice the length L and the difference between the cosine of the starting value ai of the first primary angle α and the ratio of the displacement norm A to the length L of the arm 25 is less than or equal to 1, then the range of compatible starting values is determined to be equal to the range [0, αᵢ];
if the displacement norm \vec{Δ} is strictly greater than twice the length L or the difference between the cosine of the starting value αᵢ of the first primary angle α and the ratio of the displacement norm \vec{Δ} to the length L of the arm 25 is strictly greater than 1, then the range of compatible starting values is determined as being equal to the range [0, αᵢ+δ], where δ is a small angle of predetermined value, typically comprised between 20° and 40° and for example substantially equal to 30°.

Alternatively, sub-step 1348 comprises only one of these comparisons, the choice of the range [0, αᵢ] or [0, αᵢ+δ] then depending on the result of this single comparison. Alternatively, sub-step 1348 does not comprise any comparison and the range of compatible starting values is directly determined as being equal to the range [0, αᵢ].

Sub-step 1348 is followed by a sub-step 1350 of constructing a starting configuration of the biomechanical model 100.

This sub-step 1350 comprises the selection 1352 of a starting value Bi of the second primary angle β within the range of compatible starting values.

To construct the starting configuration of the biomechanical model 100, the first primary angle α is thus assigned the starting value di and the second primary angle β is assigned the starting value βᵢ.

Sub-step 1350 is followed by a sub-step 1354 of forward integration of the estimated linear speed V on the basis of the starting configuration.

During this sub-step 1354, the processing unit 34 produces a first estimate P_{f}(tₖ) of the position of the measurement point at each sampling time tₖ of the interval by integrating the estimates of the linear speed V of the measurement point prior to time tₖ, taking as initial value an initial position Pᵢ of the measurement point at time tᵢ. This initial position is given by the following formula:

$${P}_{i} = L \cdot {Q}_{i} ( \begin{matrix}sin ( {α}_{i} ) \\ 0 \\ cos ( {α}_{i} )\end{matrix} ) + l \cdot \vec{{O}_{ι}}$$

in which:
   L is the length of the arm 25,
   l is the distance between the measuring device 20 and the elbow 28,
   \vec{Oᵢ} is the unit vector defining the direction of the forearm 22 in the inertial reference frame (Rᵢ) at the initial time tᵢ,
   Qᵢ is a rotation matrix corresponding to a rotation around the vertical axis Z converting the unit vector \vec{X} into the horizontal projection of the vector \vec{Oᵢ}.

The first estimate of the position of the measurement point is estimated at each sampling time tₖ using the following formula:

$${P}_{f} ( {t}_{k} ) = {P}_{i} + {\int }_{{t}_{i}}^{{t}_{k}} \vec{V ( t )} dt$$

Thus, a first estimate of the trajectory of the measurement point during the interval is obtained.

In parallel with this sub-step 1354, the sub-step 1340 comprises a sub-step 1355 of determining a range of finishing values of the second primary angle β compatible with the starting value βᵢ of said angle β and/or with the finishing value α_{f} of the first primary angle α.

Preferably, this sub-step 1355 comprises:
the comparison of the displacement norm \vec{Δ} with the length L of the arm 25, and
the comparison with the value 1 of the sum between the cosine of the finishing value α_{f} of the first primary angle α and the ratio of the norm of the displacement \vec{Δ} to the length L of the arm 25 (cos(a_{f})+∥\vec{Δ}∥/L).

There are then different scenarios:
if the displacement norm \vec{Δ} is less than or equal to twice the length L and the sum between the cosine of the finishing value α_{f} of the first primary angle α and the ratio of the displacement norm \vec{Δ} to the length L of the arm 25 is less than or equal to 1, then the range of compatible finishing values is determined as being equal to an angular interval extending over less than 20°, preferably over less than 15°, for example over substantially 10°, centered on an angle βₜ verifying the following relationship:

L(cos(βᵢ)−cos(βₜ))=\vec{Δ}.\vec{Z}
if the displacement norm \vec{Δ} is strictly greater than twice the length L or the sum between the cosine of the finishing value α_{f} of the first primary angle α and the ratio of the displacement norm \vec{Δ} to the length L of the arm 25 is strictly greater than 1, then the range of compatible finishing values is determined as being equal to the range [0, α_{f}+ε], where ε is a small angle of predetermined value, typically comprised between 0° and 40°, preferably between 20° and 40° and for example substantially equal to 30°.

Alternatively, sub-step 1355 comprises only one of these comparisons, the choice of the angular interval centered on the angle βₜ or the range [0, αᵢ+ε] then depending on the result of this single comparison. Alternatively, sub-step 1355 does not comprise any comparison and the range of compatible starting values is directly determined as being equal to the angular interval centered on the angle βₜ.

Sub-step 1355 is followed by a sub-step 1356 of constructing a finishing configuration of the biomechanical model 100.

This sub-step 1356 comprises the selection 1357 of a finishing value Br of the second primary angle β within the range of compatible finishing values.

To construct the finishing configuration of the biomechanical model 100, the finishing value α_{f} is thus assigned to the first primary angle α and the finishing value Br is assigned to the second primary angle β.

Sub-step 1356 is followed by a sub-step 1358 of backward integration of the estimated linear speed V on the basis of the finishing configuration.

During this sub-step 1358, the processing unit 34 produces a second estimate Pb (tₖ) of the position of the measurement point at each sampling time tₖ of the interval by integrating the estimates of the linear speed V of the measurement point subsequent to the time tₖ, taking as initial value a final position P_{f} of the measurement point at the final time tr. This initial position is given by the following formula:

$${P}_{f} = L \cdot {Q}_{f} ( \begin{matrix}sin ( {α}_{f} ) \\ 0 \\ cos ( {α}_{f} )\end{matrix} ) + l \cdot \vec{{O}_{f}}$$

in which:
   L is the length of the arm 25,
   l is the distance between the measuring device 20 and the elbow 28,
   \vec{O_{f}} is the unit vector defining the direction of the forearm 22 in the inertial reference frame (Rᵢ) at the final time t_{f},
   Q_{f} is a rotation matrix corresponding to a rotation around the vertical axis Z converting the unit vector \vec{X} into the horizontal projection of the vector \vec{O_{f}}.

The second estimate of the position of the measurement point is estimated at each sampling time tₖ using the following formula:

$${P}_{b} ( {t}_{k} ) = {P}_{f} + {\int }_{{t}_{k}}^{{t}_{f}} \vec{V ( t )} dt$$

This gives a second estimate of the trajectory of the measurement point during the interval.

Sub-step 1340 concludes with a sub-step 1359 of merging the backward and forward integrations, during which a final estimate of the position of the measurement point at each sampling time tₖ of the interval is calculated by linear interpolation of the first and second estimates:

P(tₖ)=iP_{b}(tₖ)+(1−i)P_{f}(tₖ)
where:
   P_{f}(tₖ) is the first estimate of the position obtained in sub-step 1354 by forward integration,
   P_{b}(tₖ) is the second estimate of the position obtained in sub-step 1356 by backward integration, and
   i is given by the following formula:

$$i = \frac{{t}_{k} - {t}_{i}}{{t}_{f} - {t}_{k}} .$$

This gives a final estimate of the trajectory of the measurement point during the interval for the pair of starting and finishing configurations.

From this final estimate of the trajectory of the measurement point, the processing unit 34 easily deduces a candidate trajectory of the center of gravity CG, this trajectory being composed of a set of candidate positions at time tₖ deduced from the final estimates of the positions of the measurement point by means of the following formula:

P_{G}(tₖ)=P(tₖ)−d.\vec{O(tₖ)}
in which d is the distance from the center of gravity C_{G} to the measurement point and \vec{O(tₖ)} is a unit vector defining the direction of the forearm 22 in the inertial reference frame (Rᵢ) at time tₖ, said vector \vec{O(tₖ)} being given by the following formula:

\vec{O(tₖ)}=R^{t}(tₖ)\vec{U}
where R^{t}(tₖ) is the transpose of the rotation matrix at sampling time tₖ and \vec{U} is the unit vector of the moving reference frame (Rₘ) corresponding to the axis U.

Sub-steps 1356, 1358 and 1359 are repeated for each finishing value β_{f} comprised in the range of compatible values determined in sub-step 1355 and sub-steps 1350, 1354, 1355, 1356, 1358 and 1359 are repeated for each starting value Bi comprised in the range of compatible values determined in sub-step 1348. It will be noted that, “each value comprised in the range”, means a set of discrete values spaced from each other by a predetermined step, for example substantially equal to 0.1°.

Thus, for each starting value βᵢ comprised in the range of compatible values determined in sub-step 1348 and for each finishing value β_{f} comprised in the range of compatible values determined in sub-step 1355, there is a pair of starting and finishing configurations for which a candidate trajectory of the center of gravity CG is calculated.

Returning to FIG. 5, sub-step 1340 is followed by a sub-step 1360 of evaluating a compatibility of each candidate trajectory with the biomechanical model 100.

During this sub-step 1360, the processing unit 34 evaluates a compatibility of each candidate trajectory with the biomechanical model 100. This compatibility is typically evaluated by means of a cost function which depends on the deformations that the tracking of the candidate trajectory imposes on the biomechanical model 100. Typically, the compatibility is inversely proportional to the result of the cost function: for example, in the case where the segment 106 (which corresponds to the arm 25) is extensible, the cost function can be the difference between the maximum extension of the segment 106 and the length L, the compatibility being inversely proportional to said difference. Alternatively, the cost function is constructed in such a way that the compatibility increases with the result of the cost function.

Sub-step 1360 is followed by a sub-step 1365 of selecting the candidate trajectory having the best compatibility.

During this sub-step 1365, the processing unit 34 selects the candidate trajectory whose result of the cost function reflects the best compatibility, that is to say whose result is the lowest (when the compatibility is inversely proportional to the result of the cost function) or the highest (when the compatibility increases with the result of the cost function).

Sub-step 1365 is followed by a sub-step 1370 of verifying the acceptable nature of the compatibility of the selected trajectory.

In this sub-step 1370, the processing unit 34 compares the result of the cost function of the selected trajectory with a predetermined threshold, said threshold being chosen such that the comparison of the result of the cost function of the selected trajectory with said threshold reflects the acceptable or unacceptable nature of the compatibility of the selected trajectory. Typically, said threshold is chosen such that the compatibility of the selected trajectory is considered acceptable if and only if the result of the cost function is less than the threshold (when the compatibility is inversely proportional to the result of the cost function) or greater than the threshold (when the compatibility increases with the result of the cost function).

For each interval, which will be called in the following “primary interval”, for which the compatibility of the selected trajectory is acceptable, the sub-step 1370 is followed by a sub-step 1372 of validating the selected trajectory. The selected trajectory therefore constitutes a validated estimate of the trajectory of the center of gravity CG during said primary interval.

Sub-step 1372 is followed by a sub-step 1374 of deducing a rotational component of the kinetic energy of the forearm 22 during the primary interval.

During this sub-step 1374, the processing unit 34 deduces from the rotation speed measurements during the primary interval a rotational component E_{k,r} of a mass kinetic energy acquired by the forearm 22 during the primary interval. This rotational component is typically obtained by means of the following formula:

$${E}_{k , r} = \overline{\overline{I}} {\int }_{{t}_{i}}^{{t}_{f}} \vec{ω} ( t ) \cdot \frac{d \vec{ω}}{d t} ( t ) d t$$

in which:
   I̅ is the mass inertia tensor of the forearm 22, that is to say independent of the mass of the forearm 22, expressed in the moving reference frame (Rₘ),
   \vec{ω}(t) is the rotation speed of the measurement point at a time t, expressed in the moving reference frame (Rₘ) and typically provided by the gyroscope 40, and

$$\frac{d \vec{ω}}{d t} ( t )$$

is the rotational acceleration of the measurement point at a time t, expressed in the moving reference frame (Rₘ) and typically deduced from the measurements of the gyroscope 40 by calculating the difference between two consecutive speed measurements.

Sub-step 1374 is followed by a sub-step 1376 of evaluating a primary force Wₚ exerted by the upper limb 12 during the primary interval.

During this step, the processing unit 34 evaluates said primary force Wₚ from the estimated trajectory and linear speed evolution, typically using the following formula:

Wₚ=E_{k,r}+E_{k,t}+Eₚ
in which:
   E_{k,r} is the rotational component of the mass kinetic energy acquired by the forearm 22 during the primary interval mentioned above,
   E_{k,t} is a translational component of the mass kinetic energy acquired by the forearm 22 during the primary interval, given by the following formula:

$${E}_{k , t} = {\int }_{{t}_{i}}^{{t}_{f}} \vec{{V}_{G}} ( t ) \cdot \frac{d \vec{{V}_{G}}}{d t} ( t ) d t$$

in which:
   \vec{V_{G}}(t) is the linear speed of displacement of the center of gravity C_{G} at a time t, expressed in the inertial reference frame (Rᵢ) and deduced from the estimated linear speed of the measurement point \vec{V}(t) at time t,

$$\frac{d \vec{{V}_{G}}}{d t} ( t )$$

is the acceleration of the center of gravity C_{G} at a time t, expressed in the inertial reference frame (Rᵢ) and typically deduced from the linear speed of displacement of the center of gravity C_{G} by calculating the difference between the values of said linear speed at two consecutive times.
Eₚ is a mass potential energy acquired by the forearm 22 during the primary interval, given by the following formula:

Eₚ=g(z(t_{f})−z(tᵢ))
in which:
   g is the norm of the gravitational field g
   z(t_{f}) is the vertical coordinate of the center of gravity CG at the end of the primary interval, and
   z(tᵢ) is the vertical coordinate of the center of gravity CG at the beginning of the primary interval.

Sub-step 1376 is further followed by a sub-step 1378 of determining a relationship f between primary force Wₚ and rotational component E_{k,r} of the kinetic energy acquired by the forearm 22 during each primary interval. This relationship f is typically determined by regression, preferably a linear regression, on all the pairs (primary force Wₚ, rotational component of the kinetic energy E_{k,r}) obtained for the different primary intervals.

For each interval, that will be called in the following “secondary interval”, for which the compatibility of the selected trajectory is not acceptable, the sub-step 1370 is followed by a sub-step 1382 of rejection of the candidate trajectories. None of the candidate trajectories is then considered as a valid estimate of the trajectory of the center of gravity CG during said secondary interval.

Sub-step 1382 is followed by a sub-step 1384 of deducing a rotational component of the kinetic energy of the forearm 22 during the secondary interval.

During this sub-step 1384, the processing unit 34 deduces from the rotation speed measurements during the secondary interval a rotational component E_{k,r} of the kinetic energy acquired by the forearm 22 during the secondary interval. This rotational component is typically obtained by means of the formula already mentioned for step 1374.

Sub-step 1384 is followed by a sub-step 1386 of evaluating a secondary force Wₛ exerted by the upper limb 12 during the secondary interval.

During this step, the processing unit 34 evaluates said primary force Wₛ from the rotational component E_{k,r} of the kinetic energy acquired by the forearm 22 during the secondary interval and from the relationship f between primary force Wₚ and rotational component E_{k,r} of the kinetic energy acquired by the forearm 22 determined in sub-step 1378. Typically, the processing unit 34 evaluates the primary force Wₛ by means of the following formula:

Wₛ=f(Wₚ)

Step 1300 is repeated for each time interval of the period of interest during which the linear speed of the measurement point is not a priori known and which is comprised between two phases for which the linear speed of the measurement point was determined in step 1200.

Steps 1200 and 1300 are repeated for each period of interest comprised in a predetermined period. This predetermined period typically has a duration greater than one week and, advantageously, less than three months.

After this repetition of steps 1200, 1300, the method 1000 comprises a step 1400 of determining the value of a statistical quantity, calculated on the set formed by the primary forces Wₚ and/or secondary forces Wₛ evaluated for the various time intervals comprised in the predetermined period, representative of a state of form of the individual 14 over said predetermined period.

During this step 1400, the processing unit 34 performs a statistical calculation on the set formed by the primary forces Wₚ and/or secondary forces Wₛ evaluated for the various time intervals comprised in the predetermined period. Typically, the processing unit 34 calculates an average and/or a percentile of these forces, relative to all the forces evaluated for the predetermined period. For example, the processing unit 34 calculates the 50th percentile, which corresponds to the median, the 80^{th} percentile, the 95^{th} percentile, the 99^{th} percentile, or any other percentile value of the forces evaluated for the predetermined period.

Preferably, the processing unit 34 selects the statistical quantity from a predetermined percentile range, in particular a high percentile range, that is to say percentiles greater than the 70^{th} percentile. Indeed, these high percentiles are representative of the maximum force and the maximum muscular power that the individual 14 is capable of developing, because they reflect the fastest and/or longest movements of an individual, the speed and duration of the movements of the upper limbs being constrained by the state of muscular strength. These high percentiles are therefore particularly sensitive to the state of fitness of the individual 14.

Advantageously, the statistical quantity is constituted by the 99^{th} percentile of the forces evaluated for the predetermined period.

Optionally, step 1400 comprises the selection of at least one other statistical quantity representative of a state of fitness of the individual 14 over the predetermined period.

Steps 1002 to 1400 are repeated for several predetermined periods. The processing unit 34 thus produces a set of values of the statistical quantity representative of the fitness state of the individual 14 over several predetermined periods.

Following this repetition of steps 1002 to 1400, the method 1000 comprises a step 1500 of observing the evolution of the statistical quantity across the different predetermined periods. The fitness state of the individual 14 can thus be tracked.

Advantageously, this tracking of the fitness level of the individual 14 is used to determine the effectiveness of a treatment administered to the individual 14.

Thanks to the exemplary embodiment described above, it is thus possible to track over time the physical fitness of an individual who cannot use his lower limbs, with few constraints for the individual. This tracking is particularly made possible in an uncontrolled environment and over long periods, which allows to have faithful tracking of the individual's actual fitness level.

The exemplary embodiment also allows a good estimation, in an uncontrolled environment and over a long period, of the physical forces made by an upper limb of a individual for various movements. It further allows the quality of the estimations to be improved, in particular of linear speed and position, deduced from the measurements of an inertial device, in particular when this inertial device is attached to a limb of an individual.

It will be noted that, although the above description focuses on an exemplary embodiment in which the measuring device 20 is attached to an upper limb 12 of a individual 14, the forces evaluated being those of this upper limb 12, the invention also extends to a method for evaluating the forces of a lower limb of a individual, the measuring device 20 then being attached to the leg of this lower limb. The invention also extends to a method for evaluating the forces of a forelimb or hindlimb of an animal. The person skilled in the art will easily be able to make the necessary adaptations.

1. A method for estimating a trajectory, over a displacement interval, of a point of interest belonging to a limb of an individual, the method being implemented by a data processing unit and comprising the following steps:
estimating an evolution, during the displacement interval, of a speed of a measurement point attached to the point of interest,
estimating an evolution of an orientation of the measurement point during the displacement interval, and
estimating the trajectory of the point of interest during the displacement interval from the estimated evolutions of the speed and orientation of the measurement point.

2. The method according to claim 1, wherein the individual is a human being and the limb is an upper limb of the individual.

3. The method according to claim 1, wherein estimating the trajectory comprises the following steps:
calculating several candidate trajectories by applying the estimated evolutions of the speed and orientation to different pairs of starting and finishing configurations of a biomechanical model of the limb,
evaluating the compatibility of each candidate trajectory with the biomechanical model, and
selecting the candidate trajectory having the best compatibility.

4. The method according to claim 3, wherein the point of interest is a point of a forearm belonging to an upper limb of the individual, each starting configuration of the biomechanical model of the limb being defined by a pair of starting angles formed by a first starting angle between the forearm and a vertical direction and a second starting angle between an arm of the upper limb and the vertical direction and each finishing configuration of the biomechanical model of the limb being defined by a pair of finishing angles formed by a first finishing angle between the forearm and the vertical direction and a second finishing angle between the arm and the vertical direction.

5. The method according to claim 4, wherein, for each starting or finishing configuration of the biomechanical model of the limb:
the first starting angle, respectively finishing angle, is deduced from the orientation of the measurement point at a beginning, respectively at an end, of the displacement interval, and
the second starting angle, respectively the second finishing angle, is smaller than the first starting angle, respectively the first finishing angle.

6. The method according to claim 4, wherein there is, for each value of the second starting angle smaller than the first starting angle and each value of the second finishing angle value within a predetermined interval, a pair of starting and finishing configurations for which a candidate trajectory is calculated.

7. The method according to claim 6, wherein the predetermined interval is constituted by an interval comprised between 0° and the value of the first finishing angle, or by an angular interval around an angle βₜ verifying the following relation:
$$L ( cos ( {β}_{i} ) - cos ( {β}_{t} ) ) = ( {\int }_{{t}_{i}}^{{t}_{f}} \vec{V ( t )} dt - l ( \vec{{O}_{f}} - \vec{{O}_{ι}} ) ) \cdot \vec{Z}$$
in which:
L is the length of the arm of the upper limb,
βᵢ is the value of the second starting angle in the starting configuration belonging to the same pair as the finishing configuration,
tᵢ is a start time of the start of the displacement interval,
tᵣ is an end time of the displacement interval,
\vec{V(t)} is a speed vector of the measurement point at each time t of the displacement interval,
I is a distance between an elbow of the upper limb and the measurement point,
\vec{O_{f}} is a unit vector giving an orientation of a main axis of the forearm at the end of the displacement interval,
\vec{Oᵢ} is a unit vector giving an orientation of the main axis of the forearm at the beginning of the displacement interval,
\vec{Z} is a unit vector giving the vertical direction,
the vectors \vec{V(t)}, \vec{O_{f}}, \vec{Oᵢ}, and \vec{Z} being expressed in a same inertial reference frame.

8. The method according to claim 3, wherein calculating each candidate trajectory comprises the following steps:
forward integration of the estimated evolution of the speed, taking as initial position a position of the measurement point in the starting configuration,
backward integration of the estimated evolution of the speed, taking as initial position a position of the measurement point in the finishing configuration, and
calculation of the candidate trajectory by merging the backward and forward integrations.

9. The method according to claim 1, wherein estimating the evolution of the speed of the measurement point during the displacement interval comprises the following steps:
determining an initial speed and a final speed of the measurement point respectively at a start and at an end of the displacement interval,
estimating the evolution of the speed of the measurement point during the displacement interval, said estimation comprising:
forward integration of displacement parameters of the measurement point acquired by an inertial device during the displacement interval, taking as an initial value of the speed the value of the initial speed,
backward integration of the displacement parameters, taking as the initial value of the speed the value of the final speed, and
the calculation of the estimated evolution of the speed by merging the backward and forward integrations.

10. A method for evaluating a force exerted by a limb of an individual, the method being implemented by a data processing unit and comprising the following steps:
estimating a trajectory of a point of interest belonging to the limb over at least one primary displacement interval by means of the method according to claim 1,
evaluating, from the estimated evolutions of the trajectory and speed, a primary force exerted by the limb during the or each primary displacement interval.

11. The method according to claim 10, comprising the following additional steps:
deducing, from displacement parameters acquired during the primary displacement interval, a rotational component of a primary kinetic energy acquired by at least a portion of the limb during the primary displacement interval, the primary force being a function of said rotational component of the primary kinetic energy,
determining a relationship between the primary force and the rotational component of the primary kinetic energy,
estimating an evolution of an orientation of a measurement point attached to the point of interest during at least one secondary displacement interval,
deducing, from displacement parameters acquired by the inertial device during the secondary displacement interval, a rotational component of a secondary kinetic energy acquired by said at least one portion of the limb during the secondary displacement interval,
evaluating, from the determined relationship and the rotational component of the secondary kinetic energy, a secondary force exerted by the limb during the secondary displacement interval.

12. The method according to claim 11 comprising, for each of the primary and secondary displacement intervals:
estimating an evolution, during the primary or secondary displacement interval, of a speed of a measurement point attached to the point of interest,
calculating several candidate trajectories by applying the estimated evolutions of the speed and orientation to different pairs of starting and finishing configurations of a biomechanical model of the limb,
evaluating a compatibility of each candidate trajectory with the biomechanical model, and
verifying, for each primary or secondary displacement interval, an acceptable nature of the compatibility of at least one candidate trajectory,
the trajectory of the point of interest being estimated only when at least one candidate trajectory has acceptable compatibility.

13. A method for analyzing a force exerted by a limb of an individual, the method being implemented by a data processing unit and comprising the following steps:
a) estimating, for several intervals of displacement of a point of interest of the limb occurring during a predetermined period, a force exerted by said limb by means of the method according to claim 10,
b) determining a value of at least one statistical quantity, calculated on a set formed by the estimated forces, representative of a fitness state of the individual.

14. The method according to claim 13, comprising repeating steps a) and b) for several predetermined periods and observing an evolution of the or each statistical quantity across the different predetermined periods.

15. A method for tracking a fitness state of an individual, comprising analyzing a force exerted by a limb of the individual by means of the method according to claim 13 or 14 and deducing a fitness state of the individual from said analysis.

16. A method according to claim 15, comprising administering a treatment to the individual, and evaluating an effectiveness of the treatment from the deduced fitness state.

17. An equipment comprising an inertial device for acquiring displacement parameters of a measurement point during at least one displacement interval, and a data processing unit configured to estimate, from the displacement parameters acquired by the inertial device, by implementing the method according to claim 1, a trajectory, over the displacement interval, of a point of interest attached to the measurement point.

18. A computer program product comprising code instructions for implementing the method according to claim 1 when said program is executed by a computer.

19. The method according to claim 2, wherein the point of interest is a point of a forearm of the upper limb.
